(19) United States
(12) Patent Application Publication
(10) Pub. No.: US 2024/0009335 A1
(43) Pub. Date: Jan. 11, 2024

(54) AN APPARATUS FOR STERILIZATION USING ULTRAVIOLET RAYS

(71) Applicant: WEISMACHER ECO PRIVATE LIMITED, Ahmedabad (IN)

(72) Inventors: Rajeshkumar Hariprasad ACHARYA, Ahmedabad (IN); Yusuf Nazmuddin GHADIALI, Ahmedabad (IN)

(73) Assignee: WEISMACHER ECO PRIVATE LIMITED, Ahmedabad (IN)

(21) Appl. No.: 18/253,142

(22) PCT Filed: Nov. 17, 2021
(86) PCT No.: PCT/IN2021/051076

§ 371 Date: May 16, 2023

(30) Foreign Application Priority Data
Nov. 17, 2020 (IN) 202021050024

(51) Int. Cl. A61L 2/10

(52) U.S. Cl. CPC A61L 2/10; A61L 2202/11; A61L 2202/14; A61L 2202/12

(57) ABSTRACT

The present invention discloses an apparatus for sterilization using ultra violet rays comprises roof structure (1), base structure (2) and stand (3). The roof structure (1) comprises the at least one ultra violet tube light (7) and at least one sensor (8) to detect the object placed on the base structure (2). The base structure (2) comprises the at least one ultra violet tube light (5). Said ultra violet tube lights (5) & (7) are powered by the power circuit box (6). The roof structure (1) is supported by the stand (3). The sensor (8) detects the object (4) and ultra violet tube lights (5) & (7) automatically ON and in the absence of the object (4) on platform ultra violet tube light (5) & (7) automatically OFF. All wall inside of roof structure (1) and the base structure (2) are provided with mirrors to reflect the ultra violet rays towards object (4).

### FIELD OF INVENTION

The present invention relates to an apparatus for sterilization & more particularly, it relates an apparatus that sterilize the things/object using ultra violet rays, or in other words the present invention help to prevent infectious diseases by ultra violet rays introduced from the outside and to keep things/object clean from microorganism.

### BACKGROUND OF INVENTION

One of the most concerns of the public health system across the world is controlling or reducing the spread of infectious diseases. Infectious diseases commonly spread through the direct transfer of bacteria, viruses or other germs from one person to another or through things/object. This can happen when individual touches the things where bacteria or virus are on the things. So it is required to remove/disinfect the things/object from infectious microorganism like becteria, viruses and germs.

At present, the whole world is facing an acute challenge to protect overselves from the CORONA Virus through more contactless environment and sanitization. Till the date, vaccine is not in the reach of a common person. In this direction, things are going to be automatic and contactless. A vast research is going on in this direction and some feasible solutions are obtained but these are applicable up to a certain extent.

In order to perform, sterilization, various kind of system been developed. Heretofore in one of such art is steam boil water and spray steam onto things/goods are used for disinfect. The high-temperature steam spray has the effect of removing microorganisms, but it cannot kill bacteria and viruses that have a life cycle such as endospore. With the outbreak of COVID-19, it becomes important to block the source of infection, a different method of sterilization is needed.

Hence, it is needed to invent a sterilizer that overcomes the difficulties as described above.

### OBJECT OF INVENTION

The main object of present invention is to provide an apparatus for sterilization using ultra violet rays that prevent the spread of infectious diseases by blocking external sources of infection by disinfecting goods/things.

The main object of present invention is to provide an apparatus for sterilization using ultra violet rays that disinfects all goods/things and sterilized from microorganisms such as viruses, bacteria and fungi.

Another object of present invention is to provide apparatus for sterilization using ultra violet rays that is easily to operate, easy to maintenance, less power consumption and highly efficient.

### SUMMARY OF INVENTION

The present invention relates to an apparatus for sterilization using ultra violet rays. In the present invention mainly three structures are used that is base structure, roof structure and a stand. The base structure and roof structure contains at least one ultra violet tube light. Said ultra violet tube lights are powered by the power circuit box. At least one sensor are inserted in the roof structure. When the object placed on base structure sensor detects the object and ultra violet tube lights automatically ON and disinfects the object. When there is not presence of the object on base structure sensor detects and ultra violet tube light automatically OFF. The mirrors are also assembled inside the wall of roof and base structure as a reflector of ultra violet rays. The ultra violet rays disinfect the object and sterilized from the bacteria, virus and other microorganism to block the source of infection.

### BRIEF DESCRIPTION OF DRAWINGS

Other objects, advantages and novel features of the invention will become apparent from the following detailed description of the present embodiment when taken in conjunction with the accompanying drawings.

FIG. 1 shows a schematic view of the apparatus for sterilization using ultra violet rays according to present invention.

FIG. 2 shows prospective views of the apparatus for sterilization using ultra violet rays according to present invention.

### DETAILED DESCRIPTION OF INVENTION

Before explaining the present invention in detail, it is to be understood that the invention is not limited in its application to the details of the construction and arrangement of parts illustrated in the accompany drawings. The invention is capable of other embodiment, as depicted in different figures as described above and of being practiced or carried out in a variety of ways. It is to be understood that the phraseology and terminology employed herein is for the purpose of description and not of limitation.

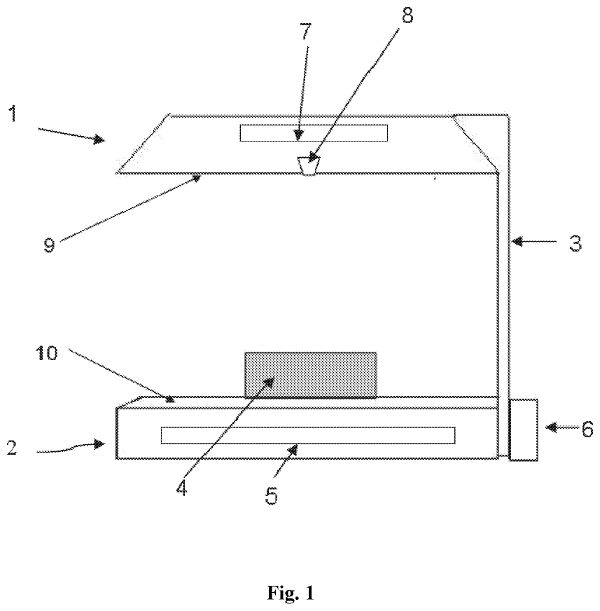

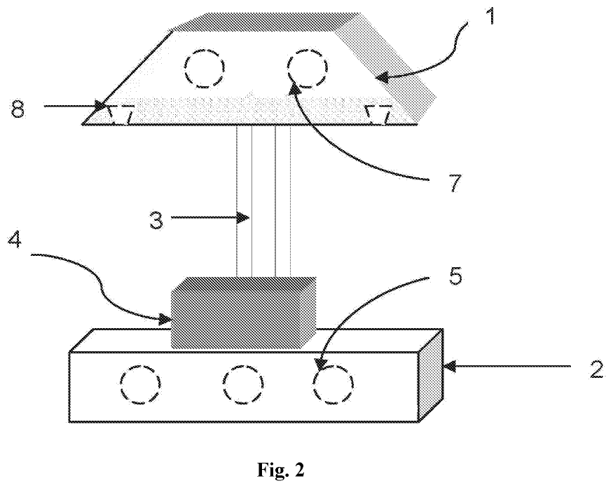

The apparatus according to FIG. 1 and FIG. 2 of the present invention comprises a roof structure (1), a base structure (2), and a stand (3) for support the roof structure (1).

An object (4) which needs to sanitization is put on base structure (2). The base structure (2) is shape of rectangular box and the roof structure (1) is trapezoidal shape. The stand (3) is mounted beside of the base structure (2) and the roof structure (1) is supported by said stand (3) as shown in FIG. 1

The Roof structure (1) mainly contains the ultra violet tube lights (7) and said tube light is powered by a power supply circuit box (6). The roof structure (1) is low perimeter side closed hollow trapezoidal structure having angular lateral sides and ultra violet tube lights (7) are mounted inside the said structure (1) due to that high radiation and whole object direction of UV rays produce. Said roof structure (1) contains mirrors or metal foil in all side walls as a reflector, so that ultra violet rays reflect to the objects (4) side. In the lower side of the roof structure (1) the transparent glass (9) is mounted. Due to that the ultra violet is passes from said glass (9). The said glass (9) is used for safety purpose.

The Base structure (2) mainly contains the at least one ultra violet tube lights (5) and said tube light is powered by power supply circuit box (6). The base structure (2) is one side closed hollow rectangular box type structure and ultra violet tube lights (5) are mounted inside the said structure. Ultra violet rays applied to lower surface of object (4) to disinfect and sterilize the object (4). Said base structure (2) contains mirrors or metal foil in all side walls as a reflector, so that ultra violet rays reflect to the objects (4) side. In the upper side of the base structure (2) the transparent glass (10) is mounted. Due to that the ultra violet is passes from said glass (10). The said glass (9) is used for safety purpose and said glass (10) makes platform for the object (4).

In the roof structure (1) plurality of a sensors (8) are used. The sensor (8) detect the presence of objects (4) being put on the base structure (2). When the sensor detect the object, the ultra violet tube light (5) & (7) are automatically ON. When the object (4) is not present on base structure (2) the ultra violet tube lights (5) & (7) automatically OFF.

The present invention is for sterilizing tableware or household good/things using ultra violet rays. Ultra violet rays having high efficiency and use for sterilization apparatus because they destroy and inactive DNA structures when irradiated to microorganisms such as virus, bacteria and fungi.

The present invention specifically provides following advantage:
Automatic operation
Low maintenance cost
Optimum usage of the UV rays
Minimum Power loss
Disinfects all goods/things and sterilized from microorganisms
Cover all portion of the goods

The invention has been explained in relation to specific embodiment. It is inferred that the foregoing description is only illustrative of the present invention and it is not intended that the invention be limited or restrictive thereto. Many other specific embodiments of the present invention will be apparent to one skilled in the art from the foregoing disclosure. All substitution, alterations and modification of the present invention which come within the scope of the following claims are to which the present invention is readily susceptible without departing from the spirit of the invention. The scope of the invention should therefore be determined not with reference to the above description but should be determined with reference to appended claims along with full scope of equivalents to which such claims are entitled.

1-6. (canceled)

7. An apparatus for sterilization using ultra violet rays mainly comprises a roof structure (1), a base structure (2), and a stand (3);
Characterized in that,
the roof structure (1) having low perimeter side closed hollow trapezoidal structure having angular lateral sides and ultra violet tube lights (7) and plurality of a sensors (8) are mounted inside the roof structure (1);
the base structure (2) having one side closed hollow rectangular box type structure and ultra violet tube lights (5) are mounted inside the said structure;
said roof (1) and base structure (2) contains mirrors or metal foil in all side wall as a reflector;
the stand (3) is mounted beside of the base structure (2) and the roof structure (1) is supported by said stand (3).

8. The apparatus for sterilization using ultra violet rays as claimed in claim 7, wherein an object (4) which needs to sanitization is put on base structure (2).

9. The apparatus for sterilization using ultra violet rays as claimed in claim 7, wherein at lower side of the roof structure (1) a transparent glass (9) is mounted.

10. The apparatus for sterilization using ultra violet rays as claimed in claim 7, wherein at upper side of the base structure (2) a transparent glass (10) is mounted.
